(19) United States
(12) Patent Application Publication
(10) Pub. No.: US 2013/0211434 A1
(43) Pub. Date: Aug. 15, 2013

(54) Latex-Free Finger Gauze Device For Cleaning And Drying The Ears

(71) Applicant: Leanne Leach, Lusby, MD (US)

(72) Inventor: Leanne Leach, Lusby, MD (US)

(21) Appl. No.: 13/765,807

(22) Filed: Feb. 13, 2013

Related U.S. Application Data
(60) Provisional application No. 61/598,632, filed on Feb. 14, 2012.

(51) Int. Cl. A61F 11/00

(52) U.S. Cl. CPC A61F 11/006
USPC 606/162

(57) ABSTRACT

A tubular, soft, absorbent, pliable, latex-free gauze device for use on the index finger to clean and dry inner and outer areas of the ear, including the folds and crevices. The device, being applied to the index finger, can be inserted into the ear only up to the auricular cartilage and does not enter the ear canal. It has sufficient size, shape, and absorbency to effectively clean and dry large areas of the ear as well as the smaller areas, including folds and crevices. The device preferably is seamed along one or both sides and the seams terminate spaced from the open end to define slits that facilitate placement of the device on a finger. The device can be used on multiple sides by rotating it on the finger.

This application claims the benefit of U.S. provisional patent application Ser. No. 61/598,632, filed Feb. 14, 2012, the disclosure of which is incorporated in full herein by reference.

### FIELD OF THE INVENTION

The present invention relates to a device for placement on a finger to use in cleaning and/or drying three areas of the human ear, namely, the outer ear, the outer interior ear (the ear wax area), and the back of the ear, including the folds and crevices. More generally, the device of the invention can be used for cleaning mammalian ears, including but not limited to humans, dogs and cats. The device does not enter the ear canal. It is latex-free and can be disposable and/or non-disposable. It can be safely and effectively used on babies, toddlers, children, and adults. It can be particularly useful and safe for cleaning the ears of pets.

### BACKGROUND OF THE INVENTION

The structure and sensitivity of the human ear presents a unique challenge for safely and effectively cleaning wax out of the ear and drying the ear because of the different shapes and types of surfaces of the ear. Many people rely upon a swab type device or a washcloth for cleaning and drying the ear. Other than drying the ears, the most common cleaning function is for the removal of ear wax.

Ear wax can become impacted and plug up the ear, causing hearing to be reduced. Rigid or semi-rigid devices are not recommended because they can push softer wax deeper into the ear canal and even against the eardrum, causing significant hearing loss and pain. Roughly 2% to 6% of the population has an ear wax impaction at any time. Water can be trapped behind these self-created wax dams.

Some prior art devices have bristles, but bristles create dryness in the sensitive ear, leading to itching. A person using a cotton-tipped swab or other rigid or semi-rigid object can scrape the ear drum or the swab can break off and become lodged in the ear canal. Also the swab can push wax deeper into the ear.

In spite of the potential dangers of inserting objects into the ear, people still buy and use swabs as a method of cleaning their ears. Pain, hearing loss, dizziness, ringing in the ear, and ruptured ear drums can be caused by inserting cotton swabs, toothpicks, pins, pens, or other rigid objects into the ear canal. No doctor will advocate inserting swabs or anything rigid or semi-rigid into the ear as a safe method for cleaning the ears. Wider or thicker swabs have been developed for use on the ears of babies but these devices are still rigid and with enough pressure can go far beyond the safe part of the ear. When these devices are being inserted, the person using them has no idea how deep the device is going into the baby's ear. Pediatricians do not advocate putting anything rigid or semi-rigid, including swabs, into the ears of babies, toddlers, or children. Hearing loss and speech loss could occur.

While a washcloth may be effective for cleaning and drying the large outer surfaces of the ear, it is not especially suitable for cleaning the outer interior ear, including the folds and crevices, and it is not an effective device for removing excess ear wax because of its thickness, size, and shape.

Another issue that is of concern with some prior art devices is the use of latex in their construction. Six percent of the general public and fifteen percent of health care workers are allergic to latex. The potential dangers of latex allergies range from sneezing and wheezing to anaphylactic shock and even death. The device of the invention is latex-free.

Applicant is not aware of any currently available device for safe and effective cleaning of the ears of pets. The present invention is particularly suited for that purpose. It does not involve the insertion of a rigid device into the ears of the pet and the injury that can consequently result. Pets, like small babies, are unable to tell the person using the device that the depth of penetration into the ear is causing pain.

U.S. Pat. No. 4,820,259 discloses a swab-like ear cleaning device that has an inflexible shaft with two enlarged sponge-like ends capable of retaining a quantity of cleaning materials which are released when the device is wet. The end pieces expand 5% to 30% when wet and generally are designed to clean the external ear only and generally would be ineffective for cleaning and drying all areas of the ear and it is rigid or semi-rigid in construction.

U.S. Pat. No. 5,807,301 discloses a device for use in cleaning the ears. The device is designed like a thimble for attachment to the end of a finger and has an extension on its end wrapped with a cotton layer. While this device is not as dangerous as the longer swabs discussed above, it is still dangerous because it has a rigid extension. Also the amount of absorbent material on the extension is so small as to be of little use, especially in effectively cleaning all areas of the ear.

U.S. Pat. No. 7,056,309 discloses a device that covers a finger to prevent the spread of microorganisms when that finger is used to clean surfaces of the body. It has an impervious layer and an absorbent layer to absorb liquids and bond to mucus. The outer layer can have antiseptic chemicals in it. The device is made of latex and is used mainly as a nasal cavity cleaning device.

U.S. Pat. No. 7,674,058 discloses a disposable finger wipe with a liquid storage application system. The device has an application side and an opposite back side and releases a liquid composition during use. It has a liquid impermeable layer that prevents the composition from migrating through the structure. It has a pouch and is stiff and is not pliable enough to clean and dry the three areas of the ear cleaned with the present invention, especially the small folds and crevice areas. Further, because of the pouch the device disclosed in this patent could not be used on both sides by turning it around on the finger.

Published US application number 2007/0299457 discloses an ear cleaning device that is designed like a swab and is inserted into the ear canal. It possesses the disadvantages of any swab-like device as discussed above.

Published US application number 2009/0112241 discloses an ear cleaning device that has an expandable end portion that can be expanded in the ear canal. The device has a shortened length and would not pose the dangers of longer swab-like devices discussed above, but it has brush-like filaments or bristles on the ear-engaging end portion which has a tendency to dry out the ear and make them itch. It would have very little utility in cleaning and drying the ear, especially larger surfaces such as behind the ear.

Published international application number WO-2003/043528 discloses an ear cleaning device that is held to the end of a finger by flexible legs 14 and has a protruding end 20 for cleaning surfaces of the ear. The device is made of a material such as PVC or polyurethane foam. It is designed for cleaning the ear canal only, which is a safety hazard.

Published international application number WO-2011/062858A2 discloses a conically shaped device for attachment to the end of a pinky finger and has a point that protrudes into the ear. The conical shape would make the device difficult to keep in place on the end of the finger during use. Its shape would not allow it to effectively clean or dry the three areas of the ears cleaned and dried with the present invention. The device in this application is designed to clean inside the ear.

None of the above-mentioned prior art devices can safely and effectively clean and dry all three areas of the ear, namely, the outer ear, the outer interior ear (the ear wax area), and the back of the ear, including the folds and crevices. The prior art devices lack the necessary shape, structure, softness, pliability, and absorbency, which limits their use. Some of the above-mentioned devices have safety issues and/or potential adverse side effects from allergic reactions to latex. Some of the prior art devices are rigid or semi-rigid and are not recommended by doctors for this type of use. The present device has none of these issues. The primary object of the present invention is to provide a safe, effective, and convenient means of cleaning and drying the ears of mammals, notably humans and animals such as dogs and cats, without the short-comings of a thick wash cloth or dangerous swabs.

### SUMMARY OF THE INVENTION

The ear cleaning device of the present invention is made of thin, latex-free, absorbent gauze and comprises an elongate tubular body with a closed distal end and an open proximal end. It is easily transportable and can be carried in a pocket or purse much more unobtrusively than a washcloth. It is convenient to take camping or on trips or during hospital stays, and the like. It can be effectively used to clean ear wax from the ear and to clean and dry the large surface areas as well as the folds and crevices of the ear. As used herein, the term “ear” is intended to encompass mammalian ears including humans, dogs, cats, and other animals and has equal applicability in the home or in veterinarian clinics, pet grooming and boarding facilities, and the like.

The body preferably tapers over at least a portion of its length to a reduced diameter at its distal end to ensure a close fit on the end of the finger and enable the end of the device to enter the small areas and crevices of the ear. At least one seam extends along the length of the body from one end to the other, and a slit extends from the end of the seam to the adjacent open end of the body, permitting the open end to be flared outwardly to facilitate placement of the device on the finger. In a preferred embodiment, a seam extends along each of two opposite sides of the body and a slit extends from the end of each seam to the open end. The slits at the open end of the device are formed by leaving unattached the seam or seams along the sides. The slits provide a wider entry into the open end to more easily accommodate the finger. Reinforcement preferably is provided at the end of the seam at the base of the slit. Although the device is intended primarily for use on the index finger, it could be placed on any finger, including the thumb, which probably would be the preferred method for cleaning and/or drying the ears of animals such as cats, for example.

As used herein, the term “gauze” is intended to refer generally to a thin fabric and is not intended to be limited to the transparent open weave material commonly thought of as comprising gauze, although that material is intended to be included in the term “gauze” as used herein. The “gauze” of the invention may be made of many materials, and preferably is made of a latex-free absorbent gauze material that may be either woven or nonwoven and bleached or non-bleached, sterile or non-sterile, and disposable or non-disposable. It can be made of, but is not limited to, cotton, polyester, rayon, cellulose, polyurethane, polyethylene, polyvinyl chloride and other various co-polymers. It can be made of one ply or multiple plies and can be used on multiple sides if needed or desired by rotating it on the finger. It can be used damp, wet with water, or dry.

The device is designed to be used on any finger, including the thumb, but preferably on the index finger, so it does not go into the ear any further than the auricular cartilage, making it safe for cleaning ear wax from the ear. In a preferred construction the device has a length adapted to extend from its closed end at the tip end of the finger to its open end at about the first knuckle or joint spaced from the base of the finger. It is stretchable in a transverse direction so that it can fit snugly on the finger and be reliably held in position during use.

The device is particularly safe and effective for cleaning and drying the ears of mammals, notably humans and animals such as dogs and cats, without the short-comings of a thick wash cloth or dangerous rigid or semi-rigid swabs.

The closed end of the device can either be folded over or seamed, and the seams extending along each side of the device can made with, but are not limited to, thread, tape, air bonding, ionic bonding, thermal bonding, ultra sonic bonding, resin bonding, chemical bonding or hydro entanglement bonding.

In one embodiment of the invention a first portion of said body is tapered from approximately a midportion thereof to a reduced diameter at its closed end, and a second portion extending from said first portion to the open end is substantially cylindrical and of substantially uniform diameter. The cylindrical portion preferably is adapted to fit on that area of the finger extending approximately between the first and second knuckles of the finger although it could extend onto the area between the first knuckle and the base of the finger.

In another embodiment of the invention, the device tapers substantially uniformly over its whole length. The device according to this form of the invention generally is intended to be non-disposable but is not necessarily so.

Either device according to the invention can be disposable or non-disposable, and sterile or non-sterile. It is not limited to one size fits all, although it does accommodate various sizes of index fingers as a result of its construction.

The gauze device of the invention is soft, absorbent, and pliable so that it may be effectively used to clean and dry the outer, outer interior (the ear wax area), and back of the ear, including the folds and crevices. The product can be safely used on babies, toddlers, children and adults. It also has particular utility in the cleaning and drying of the ears of animals such as dogs and cats, for example.

The non-disposable device can be made more dense, longer, and wider in diameter than the disposable device, and it can be washed and dried.

The disposable gauze has the ability to stretch in a transverse direction, and the non-disposable gauze can be stretchable or non-stretchable, depending upon the gauze used.

The invention effectively cleans more areas and surfaces of the ear, including folds and crevices, than any other product, including swabs, which are too small to effectively clean large surface areas of the ear and cannot effectively dry the large surface areas. Wash cloths generally are too large to clean and/or dry the small areas such as the folds and crevices and interior ear areas.

### BRIEF DESCRIPTION OF THE DRAWINGS

The foregoing, as well as other objects and advantages of the invention, will become apparent from the following detailed description when taken in conjunction with the accompanying drawings, wherein like reference characters designate like parts throughout the several views, and wherein:

FIG. 1 is an isometric view of the disposable finger gauze device of the invention.

FIG. 2 is a side view in elevation of the disposable device of FIG. 1.

FIG. 3 is a side view in elevation of the non-disposable finger gauze device of the invention.

FIG. 4 is a side view depicting placement of the finger gauze device on the index finger.

FIG. 5 is a top view showing the finger gauze device on the index finger.

FIG. 6 is a schematic view showing the three parts of the ear, namely, the outer ear, the middle ear, and the inner ear.

FIG. 7 is a schematic view of the outside areas of the ear and the folds and crevices that are cleaned and dried with the device of the invention.

FIG. 8 illustrates the back areas of the ear and the folds and crevices that are cleaned and dried with the device of the invention.

FIG. 9 illustrates the safe application of the present finger gauze device in the ear.

FIG. 10 illustrates the unsafe application of swabs or rigid, semi-rigid, or similar objects when they are inserted too far into the ear.

FIG. 11 illustrates how swabs or rigid, semi-rigid or similar objects push wax further into the ear when they come in contact with wax in the ear.

FIG. 12 illustrates what commonly happens when swabs or rigid, semi-rigid, or similar objects break off in the ear canal.

FIGS. 13 and 14 depict the device of the invention being used to clean and/or dry the ears of a cat.

### DETAILED DESCRIPTION OF THE PREFERRED EMBODIMENTS

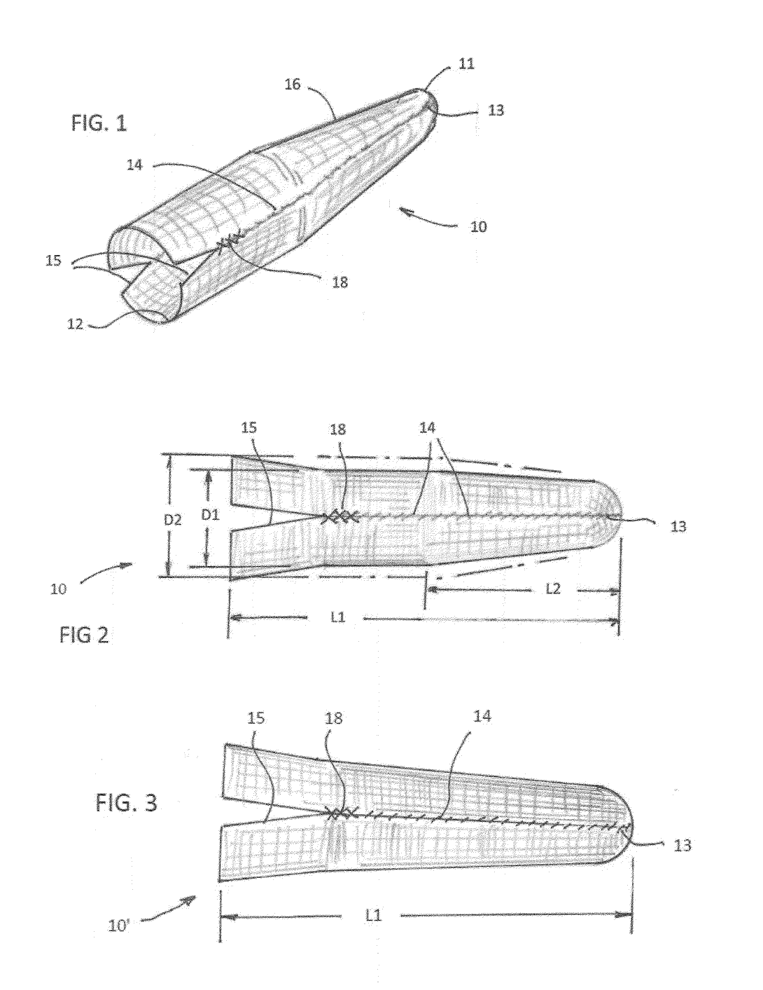

One embodiment of the finger gauze device of the invention is shown at 10 in FIGS. 1-4. The device 10 is made of soft, absorbent, pliable gauze, which can be either woven or nonwoven and bleached or non-bleached. It can be made of one ply or more and if needed can be used on more than one side by rotating it on the finger. It can be used damp, wet with water, or dry. It preferably is made of a material selected from cotton, polyester, rayon, cellulose, polyurethane, polyethylene, polyvinyl chloride or other co-polymer, but is not limited to those materials. It comprises a tubular body having a closed end 11 and an open end 12. The closed end can be folded over or seamed at 13, and a soft seam 14 extends along one or both sides. The seam or seams 14 terminate short of the open end, defining two slits 15 extending a short distance along the length of the device from the end of the seam to the open end. The slits 15 facilitate application of the device to the finger, and also more readily accommodate different sizes of fingers. The device preferably is reinforced at 18 at the inner ends of the slits.

The device 10 shown in FIGS. 1 and 2 is tapered at 16 over approximately half its length L2 to a reduced diameter at its closed end 11, and is cylindrical and of substantially uniform diameter over the remainder of its length toward the open end 12. The tapered area 16 makes the device more accessible to all areas of the ear, and the cylindrical portion extends over and fits snugly on approximately the area between the first and second knuckles of the finger. The unstretched diameter of the open end 12 is indicated by D1 and the stretched diameter indicated by D2. The gauze preferably fits snugly on the finger and stretches transversely ¼ inch or more to accommodate different sizes of fingers.

A second embodiment of finger gauze device according to the invention is shown at 10′ in FIG. 3. In this form of the invention, the device tapers substantially uniformly throughout its length L1 from the closed end to the open end. Otherwise, and except as described below, it is essentially the same as the device 10 previously described.

If the device is intended to be disposable, it preferably has a length L1 of from about 1 inch to about 4 inches and a diameter D1 of from about ½ inch to about 1½ inches, tapering from a larger diameter at approximately its midportion to a smaller diameter at its closed end, and the slits 15 preferably have a length of from about ½ inch to about 1 inch, depending upon the overall length of the device. It should be understood, however, that the device is not limited to these dimensions.

If the device is intended to be non-disposable, it may be slightly larger than the disposable device, and preferably has a length L1 of from about 1 inch to about 5 inches, a diameter D1 of from about ½ inch to about 1¾ inches, and the slits have a length of from about ½ inch to about 2 inches. The device also tapers gradually over substantially its entire length to a reduced diameter at its closed end.

It should be understood that the device is not intended to be limited to the stated dimensions, but could have other dimensions as desired or necessary. Also, the gauze in the non-disposable device can be denser than that used in the disposable device. The non-disposable device is intended to be reused and can be washed and dried. These relative sizes depend on the gauze used.

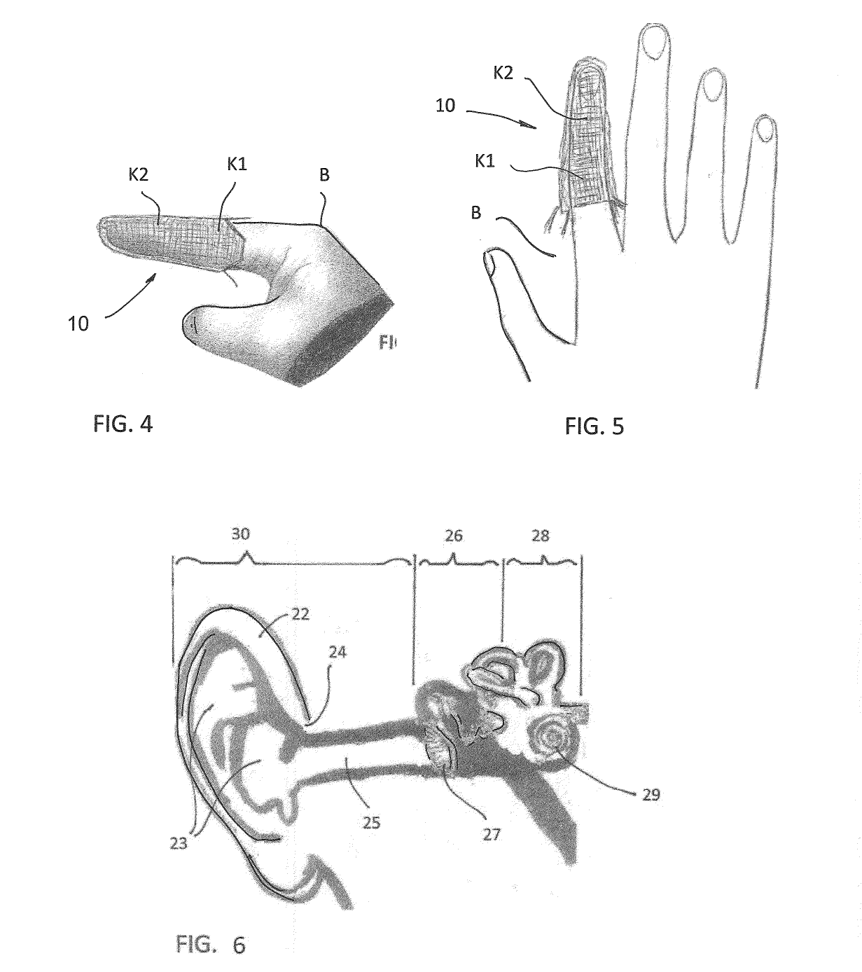

The devices 10 or 10′ according to the invention are shown in operative position on an index finger in FIGS. 4 and 5. As depicted in these figures, the open end of the device extends only to or slightly beyond the first knuckle K1 spaced from the base B of the finger. In that form 10 tapered over only about half its length as shown in FIGS. 1 and 2, the tapered portion extends from the second knuckle K2 to the tip end of the finger, and the non-tapered cylindrical portion extends essentially between the first and second knuckles.

FIG. 6 illustrates the three parts of the ear discussed in relation to the present invention, i.e., the outer ear 30, the middle ear 26, and the inner ear 28. The outer ear picks up and amplifies sound and extends from the front auricle 22 through the outer interior 23 and past the auricular cartilage 24 into the ear canal 25. The middle ear 26 transfers sound waves from the ear to the fluid of the cochlea and includes the ear drum 27. The inner ear 28 includes the cochlea 29, which transfers the vibrations into neural signals.

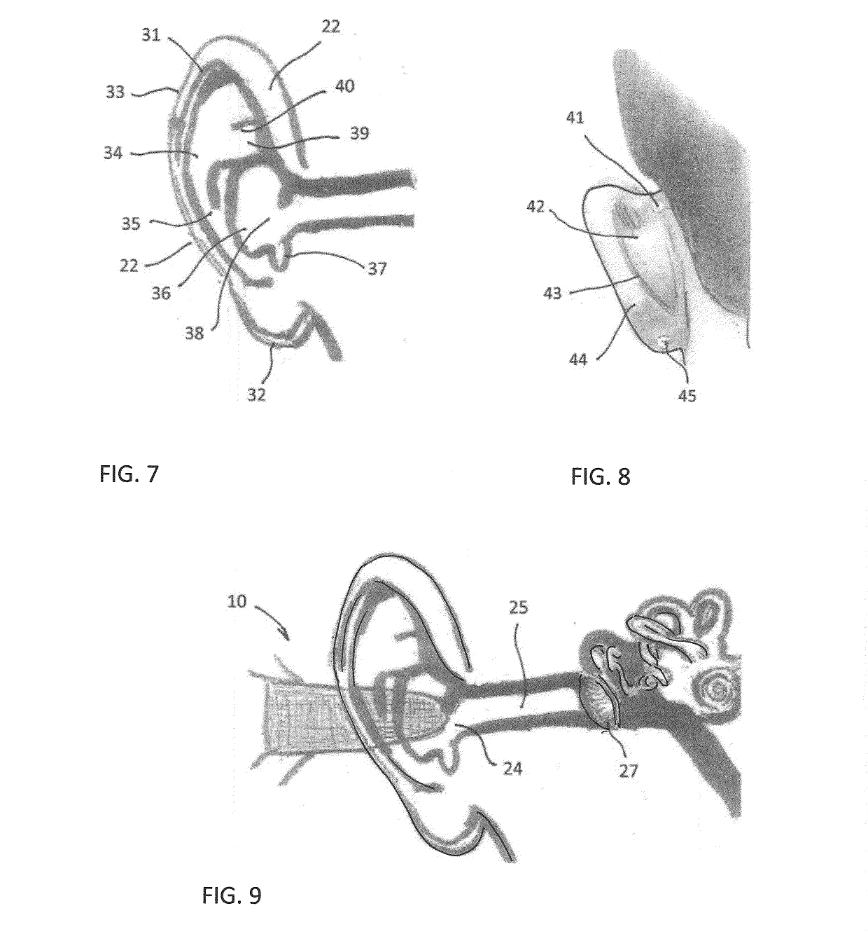

As shown in FIG. 7, the front areas of the outer ear that are cleaned and dried with the gauze device 10 or 10′ of the invention include the folds and crevices 31, the auricle 22 and the frontal lobe 32. The outside interior areas cleaned and dried by the device of the invention include the scapha 33, antihelical fold 34, antihelix 35, antitragus 36, tragus 37, external auditory meatus 38, concha 39, fossa 40, and the auricular cartilage, the ear wax area cleaned without entering the ear canal 25.

As shown in FIG. 8 the back areas of the outer ear that are cleaned and dried with the device 10 or 10′ of the invention include the folds and crevices 41, the body 42 of the back of the ear, the folds 43 of the back of the ear, the back auricle 44 of the ear, and the back 45 of the ear lobe.

FIG. 9 illustrates the safe application of the device of the invention in the ear. As shown, the device can be inserted only up to the auricular cartilage 24, which is as far as the index finger can reasonably go into the ear. The device does not enter the ear canal 25 and never comes in contact with the ear drum 27. The device of the invention does not push wax further into the ear and does not break off or get lodged inside the ear as can happen with some prior art devices.

FIGS. 10-12 all illustrate what can happen when swabs or other narrow rigid objects are inserted into the ear canal. As a result of these potential problems, emergency situations arise. The danger is increased when a swab or rigid object is inserted into the ear of a baby or toddler since the person using the device will not know when the object has been inserted too far. Disabilities such as hearing loss and speech loss could occur.

FIG. 10 illustrates what happens when a swab or other object 46 of rigid or semi-rigid material protrudes into the ear too far past the auricular cartilage 24 and into the ear canal 25, hitting, scraping or puncturing the ear drum 27.

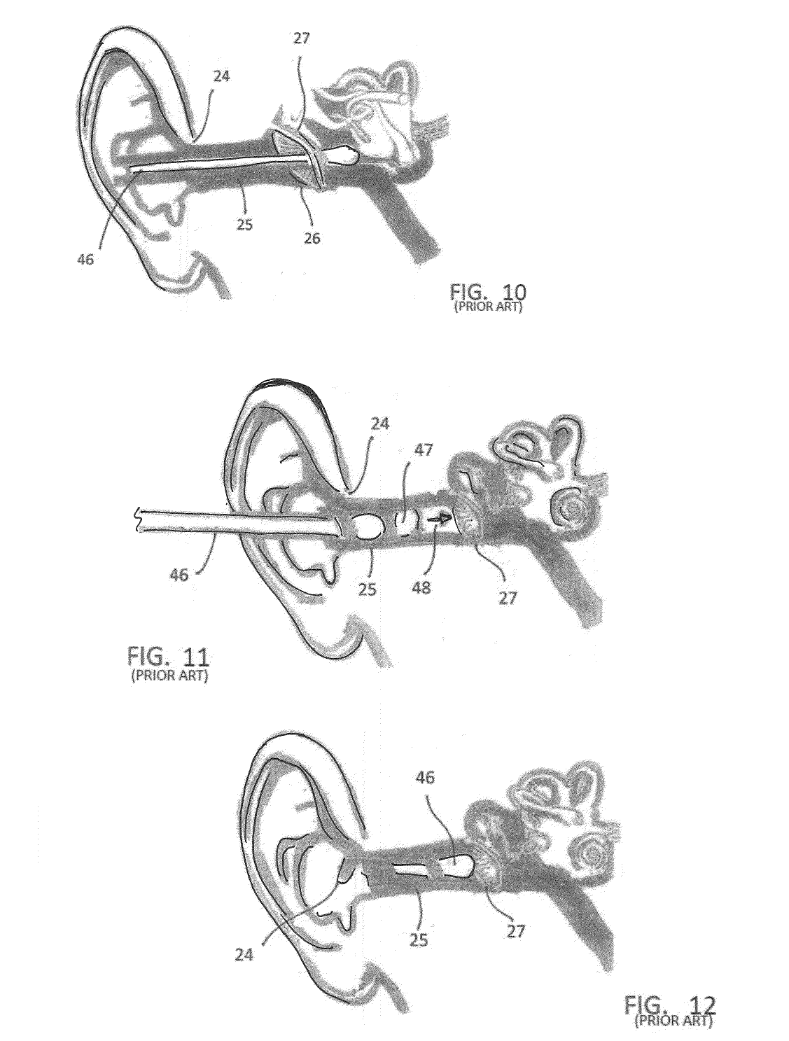

FIG. 11 illustrates what happens when a swab or other object 46 of rigid or semi-rigid material passes through the auricular cartilage 24 and enters into the ear canal 25 where it comes in contact with ear wax 47. The arrow 48 illustrates the swab pushing the wax further into the ear canal toward the ear drum 27, causing impaction, pressure and pain.

FIG. 12 illustrates what happens when a swab or other object 46 of rigid or semi-rigid material breaks off and gets lodged in the ear canal 25, potentially hitting the ear drum 27, causing bleeding and extreme pain.

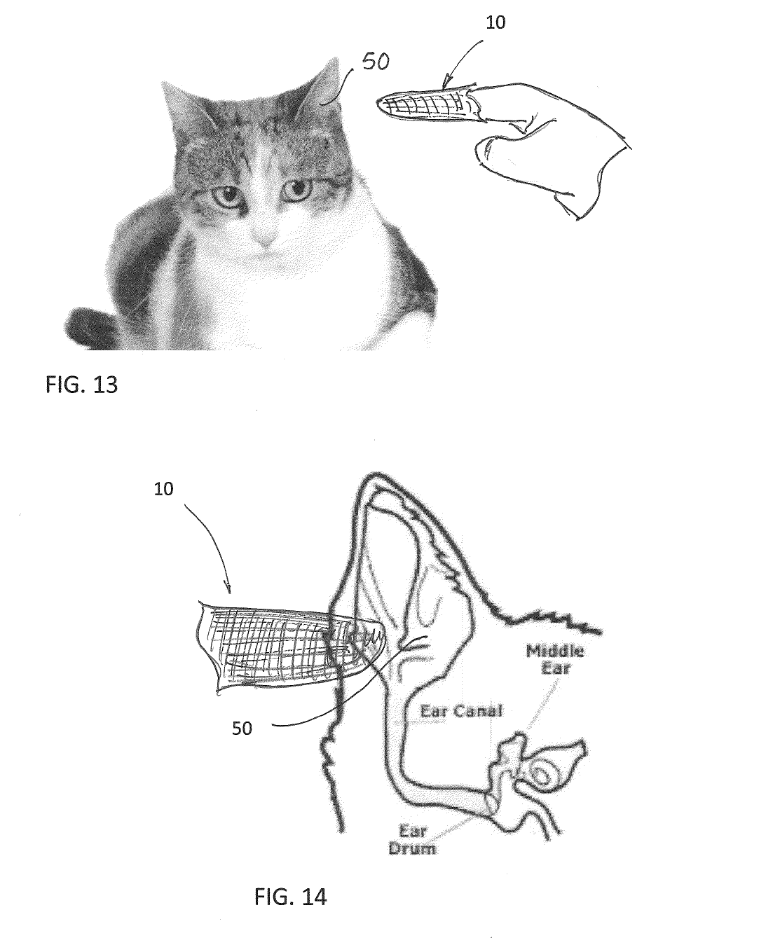

FIGS. 13 and 14 depict the device 10 being used to clean and/or dry the ears of a cat. The device is especially suited to clean and/or dry the inner areas 50 of the ear, as compared with using a washcloth or similar device as necessary in the prior art.

The features and advantages of the invention have been described in relation to the human ear, but the invention is equally applicable to cleaning and drying the ears of animals such as dogs and cats and the same comments as applied to humans generally apply to animals.

While particular embodiments of the invention have been illustrated and described in detail herein, it should be understood that various changes and modifications may be made in the invention without departing from the spirit and intent of the invention as defined by the appended claims.

What is claimed is:

1. A soft, absorbent, pliable, latex-free gauze device for application to the finger of a human hand to clean and dry inner and outer areas of a mammalian ear, wherein said device comprises:
a tubular body having a proximal open end and a closed distal end;
a seam extending longitudinally along at least one side of the body from the closed end toward the open end and terminating short of the open end to define a slit extending a short distance along the body from the end of the seam to the open end; and
at least a portion of said body being tapered to a reduced diameter toward said closed end.

2. A device as claimed in claim 1, wherein:
said body is reinforced at the juncture of said seam and slit.

3. A device as claimed in claim 2, wherein:
a first portion of said body is tapered from approximately a midportion thereof to a reduced diameter at its closed end, and a second portion extending from said first portion to the open end is of substantially uniform diameter.

4. A device as claimed in claim 2, wherein:
said body is substantially uniformly tapered over substantially its entire length.

5. A device as claimed in claim 3, wherein:
a seam extends along each of two opposed sides of the body from the closed end toward the open end, said seams terminating short of the open end to define a slit extending a short distance along the body from the end of each seam to the open end.

6. A device as claimed in claim 4, wherein:
a seam extends along each of two opposed sides of the body from the closed end toward the open end, said seams terminating short of the open end to define a slit extending a short distance along the body from the end of each seam to the open end.

7. A device as claimed in claim 1, wherein:
said body has a length adapted to extend only from the tip end of the finger to about the first knuckle.

8. A device as claimed in claim 3, wherein:
said body has an overall length adapted to extend only from the tip end of the finger to about the first knuckle, said first portion being adapted to fit closely on that portion of the finger extending from the second knuckle to the tip end, and said second portion adapted to fit closely on that portion of the finger extending between the first and second knuckles.
